(19) United States
(12) Patent Application Publication
(10) Pub. No.: US 2015/0216711 A1
(43) Pub. Date: Aug. 6, 2015

(54) Protective Adhesive Safety Strips

(71) Applicant: Joe Peck, Portland, OR (US)

(72) Inventor: Joe Peck, Portland, OR (US)

(21) Appl. No.: 14/586,145

(22) Filed: Dec. 30, 2014

Related U.S. Application Data
(63) Continuation-in-part of application No. 14/169,466, filed on Jan. 31, 2014.

(51) Int. Cl. A61F 5/08; B32B 5/02; B32B 27/08; G08B 5/36; B32B 7/12; B32B 27/32; B32B 27/40; A61M 35/00; B32B 27/36

(52) U.S. Cl. CPC A61F 5/08; A61M 35/00; B32B 5/022; B32B 27/08; B32B 27/36; B32B 7/12; B32B 27/32; B32B 27/40; G08B 5/36; B32B 2255/10; B32B 2255/26; B32B 2307/51; B32B 2535/00

(57) ABSTRACT

A strip device for improving visual safety or for providing medicaments comprising a first layer comprising any combination of a reflective layer, a medicament layer, or both, and a constructed truss member consisting of an adhesive backing, an adhesive layer, a resilient layer, and a cover layer coupled to the adhesive layer and whereby the resilient layer is disposed between the cover layer and the adhesive layer.

### PRIORITY CLAIM

The present application is a Continuation-in-Part application of co-pending utility patent application Ser. No. 14/169,466 filed on 2014 Jan. 31 by the common inventor. The present invention claims priority to this invention for all purposes and further incorporates this application by reference as if fully set out herein.

### FIELD OF THE INVENTION

The present invention relates to functional safety devices, specifically devices worn directly on the skin or over the clothing of a wearer or on an item such as a bicycle or helmet, or such, and the present invention's device includes highly visible features to improve visibility and therefore safety of pedestrians, runners, cyclists, an other sports or leisure activities, for example.

### BACKGROUND

Strips, such as external nasal dilators, include an adhesive adapted for use directly in contact with human skin: These known adhesives provide a secure bond between the strip body and the skin, yet allow the user to remove the strip without damaging the skin or causing pain. Such strip bodies include a resilient member that is adapted to lift the tissue adjacent to nasal passages and in so doing, enable the wearer of the strip to breathe easier. While such devices improve breathing, there has been little attention to other improvements to the strip to improve visibility of the wearer and to provide medicaments to the user by impregnating the strips with a predetermined medicinal or herbal layer that can then infuse the tissue with the medicament to provide, for example, pain relieve, reduce swelling, or otherwise improve the comfort of the user.

Other devise, such as reflective tape, adhere to garments or gear to provide higher visibility to the item or wearer in low light conditions, but are ill-suited to adhere directly to the skin of the wearer without causing harm or damage to the wearer. Other limitations of known reflective tape devices include the inability to treat pain, provide medicaments, or to improve breathing, if worn over the bridge of the nose.

External devices configured to aid in expanding nasal passages are well-documented in the art. Many such devices include one or more layers that include a resilient truss member configured to pull the external tissue around the nasal passageways in an effort to open the nasal passages. Ierulli in U.S. Pat. No. 8,444,670 issued on 2013 May 21 describes one exemplary device. Therein Ierulli describes a nasal dilator consisting of a laminate of vertical layers to form a truss adapted to engage the outer wall tissues and traverse the bridge of a wearer's nose.

Other examples in the art of external nasal dilators teach a narrower segment configured to bridge the nose and broader portions to arrange on the external tissue above the nasal passages. Some examples of this include U.S. Pat. No. 6,244,265 issued to Cronka et al. on 2001 Jun. 12; U.S. Pat. No. 8,062,329 to Ierulli on 2011 Nov. 22; and U.S. Pat. No. 5,476,091 to Johnson on 1995 Dec. 19, for example.

Yet others teach a deep cleft adjacent to the bridge of the wearer's nose, for example U.S. Pat. No. 8,047,201 issued to Guyuron et al. on 2011 Nov. 1.

And yet others teach a broader segment at the bridge of the wearer's nose. For example, U.S. Pat. No. 8,342,173 to Lockwood, Jr. on 2013 Jan. 1 includes a convex protrusion located near the bridge of the wearer's nose to indicate the proper orientation of the device.

Despite the previous attempts to provide improved breathing from external nasal dilator devices, there remains a need for an improved device that is comfortable to wear, enhances nasal breathing, and improves adhesion to the external tissue while also maximizing the contact area to impart greater outward force on the internal nasal passages to enable reduced restriction of the nasal pathway.

### SUMMARY OF THE INVENTION

The present invention in various preferred embodiments improves upon and otherwise overcomes limitations in the art.

One use of the present invention includes highly visible strips that, in one preferred embodiment, configure for use as nasal dilators that not only aid in making the wearer more visible in low-light conditions (for example, a night-time jogger or runner), but also function to improve breathing. In other embodiments, the strips configure for use as passive safety strips that adhere to clothing, footwear or a user's skin directly and safely. In yet other contemplated and preferred embodiments the present invention includes active safety elements including light-emitting diodes (LED) with a self-contained power source (either batteries or rechargeable solar-powered cells) to provide self-powered illumination to improve wearer safety in low-light conditions. Other active safety elements include medicaments infused into a layer of the strip whereby the layer contacts the skin of the wearer.

Advantages of the present invention include protective adhesive safety strips so wearers can be seen in low-light conditions. Accordingly, the invention enables safety in a myriad of uses and conditions such as a wearer running or walking along side of a dark or poorly lit road, in the woods, or in any common darkened area. Other features include the ability to illuminate in dark areas for hours providing safety, strong adhesive adheres to skin, shoes, clothing, bicycles and bicycle helmets, power saving LED feature is reusable for 2-3 days, impervious to rain, sleet, snow, and mud, for example.

Some other contemplated uses include, but are not limited to, adhering to visible areas of the skin for running and walking shoes, caps, safety helmets, any parts of apparel, dog leashes and collars, glow caps and glow helmets made from the customized materials.

The adhesive, applied to a self-adhesive layer and under a removable protective (non-adhesive) layer enables a user to adhere the strip directly to skin, clothing, footwear, or other gear, by removing the protective (backing sheet) layer and sticking directly to the skin or item of interest.

The strips configure in a myriad of shapes and sizes. For example, some strips target adoption by children and would include fun shapes, colors, and sizes. Other strips target sports activities and are a functional safety device. They have active pain ingredients added to provide pain relief. The glow effects are for safety.

Some uses include reflective devices adapted for use as nasal dilators or visual-safety devices applied to the skin or gear or clothing or footwear for activities including, running and walking shoes. Some clothing or gear or footwear would include, but is not limited to, caps, visors, dog and cat collars and leashes, bicycles (frames, outside of tires, plates), and helmets. For skin applications for non-nasal dilators, the strips can be applied to the forehead, back of the neck area, legs and arms. For shoe applications, the strips can be applied to the tip and heel. For cap/visor/helmet applications, the strips can be applied to the front and back areas.

Larger sized strips or patches in other contemplated embodiments would be useful for skin applications for the body (chest and back areas), caps, visors, helmets, apparel, home and business (front and rear doors), mailboxes, bags (shopping, trick-or-treat, etc.)

The various embodiments include functional glow-in-the-dark safety strips and patches. They are made from comfortable non-irritating materials. A pigment is imbedded into the coating of the sheets to provide the glowing effects for nighttime safety and is “recharged” from natural and other light sources without batteries. The illuminating effects will last for several hours in dark areas, and provides safety from a distance for people and their pets who run, walk, bike, and play at night. This method of safety is an improvement over reflective devices that often get damaged, crack, and require light to approach from shorter distances to work more efficiently. Strips and patches are more cost effective for consumers, and are impervious to rain, mud, sleet and snow, to function and perform without any interruption to ensure safety.

There will be solid and imprinted variations. For the imprints, the decoration or logo will be imprinted on the front of the material and will glow in the dark.

Glow safety nasal dilator with glare guard protection. Two strips, one of each side of the glow nasal dilator, will extend at each end of the nasal dilator and will fit under each eye. The strip extensions will be a dark color and will have a protection film that eliminates glare. The user gets the benefits from improved breathing and safety effects from the nasal dilator, and the protection from glare.

Other embodiments discussed in the parent application include:

Type I—One preferred embodiment of a nasal dilator for reducing the propensity of nasal passages from contracting during breathing, the dilator comprises: a constructed truss member having a normally substantially planar state, the constructed truss member comprising a plurality of layers, the layers comprising an adhesive backing layer; an adhesive layer removably coupled to the adhesive backing layer; a resilient layer coupled to the adhesive layer; and a cover layer coupled to the adhesive layer whereby the resilient layer is disposed between the cover layer and the adhesive layer. And, further whereby an at least one of the adhesive backing layer, the adhesive layer, or the cover layer is symmetrical with respect to a common centerline and further comprises a top edge having a first notch arranged substantially horizontally along the top edge and disposed perpendicular to the common centerline, the first notch intermediate to and connecting to an upwardly and outwardly diverging first-left and first-right portion of the top edge, a bottom edge disposed opposite the top edge, the bottom edge having a second notch arranged substantially horizontally along the bottom edge and disposed perpendicular to the common centerline, the second notch intermediate to and connecting to an downwardly and outwardly diverging second-left and second-right portion of the bottom edge, and a left and right side portion connecting the top and bottom edges, each left and right side portion comprising at least two arcuate line member segments coupled together, the at least two arcuate line member segments connecting between the top edge and the bottom edge.

Type II—Another preferred embodiment of a nasal dilator, similar to the Type I preferred embodiment, but wherein the resilient layer further comprises a resilient member, the resilient member being symmetrical with respect to a centerline of the resilient member, the resilient member forming a substantially ovoid profile, the ovoid profile being a first height at the centerline and converging to common left side intersection point and a common right side intersection point with respect to the centerline.

Type IIA—Another preferred embodiment alters the Type II embodiment slightly whereby the resilient member further includes an ovoid profile further having a length that is substantially from about three to about four times longer than the first height.

Type IIB—Another preferred embodiment alters the Type II embodiment slightly whereby the resilient member includes an ovoid profile further having a length that is substantially from about four times to about five times longer than the first height.

Layer 2, Type C—The type 1 embodiment is further configured in a new embodiment of nasal dilator wherein the resilient layer further comprises a resilient member, the resilient member being symmetrical with respect to a centerline of the resilient member, the resilient member comprising, with respect to the centerline, a top comprising a top-first side and a top-second side, both the top-first and top-second side diverging upward and outward from the centerline and a top-flat segment configured substantially horizontal and perpendicular to the centerline, the top-flat segment configured intermediate to both the top-first and top-second sides, the resilient member further comprising a bottom disposed opposite from the top, the bottom comprising a bottom-first side and a bottom-second side, both the bottom-first and bottom-second side diverging downward and outward from the centerline and a bottom-flat segment configured substantially horizontal and perpendicular to the centerline, the bottom-flat segment configured intermediate to both the bottom-first and bottom-second sides, the resilient member further comprising a left side extending from the top-first end to the bottom-first end, the left side comprising an arcuate line segment having a first-convex segment adjacent to the top-first side, an intermediate concave segment and an upward-sweeping second-convex segment connecting to the bottom-first side, and the resilient member further comprising a right side extending from the top-second end to the bottom-second end, the right side comprising an arcuate line segment having a first-convex segment adjacent to the top-second side, an intermediate concave segment and an upward-sweeping second-convex segment connecting to the bottom-second side.

LAYERS 1, 3, 4 TYPE I—The Type I nasal dilator further configures so that each one of the adhesive backing layer, the adhesive layer, or the cover layer is, individually and respectively, symmetrical with respect to a common centerline and further comprises a top edge having a first notch arranged substantially horizontally along the top edge and disposed perpendicular to the common centerline, the first notch intermediate to and connecting to an upwardly and outwardly diverging first-left and first-right portion of the top edge, a bottom edge disposed opposite the top edge, the bottom edge having a second notch arranged substantially horizontally along the bottom edge and disposed perpendicular to the common centerline, the second notch intermediate to and connecting to an downwardly and outwardly diverging second-left and second-right portion of the bottom edge, and a left and right side portion connecting the top and bottom edges, each left and right side portion comprising at two arcuate line segments connected together to connect to the top edge and bottom edge.

Layers 1, 3, 4—The Type II nasal dilator further configures such that at least one of the adhesive backing layer, the adhesive layer, or the cover layer is symmetrical with respect to a common centerline and further comprises a left side indent and a right side indent each indent respectively disposed on the corresponding left side or right side portion and further wherein the corresponding left side and right side indent is disposed at a base of an intermediate concave section disposed between each one of the at least two arcuate line member segments of the left side portion or right side portion and wherein the at least two arcuate line member segments consists of two concave line arcuate line member segments having an intermediate convex section there-between.

Type III—Yet another embodiment of a nasal dilator includes the Type I dilator modified so that the top edge first notch is an elongated notch; the bottom edge second notch is an elongated notch; and the left and right side portion connecting the top and bottom edges further comprises at least four concave line segment members adjacently arranged and configured to link the top edge and bottom edge.

Type IIIA—The Type III nasal dilator is further adapted whereby the left and right at least four concave line segment members consists of four concave line segment members respectively on the left side portion and the right side portion; a left slot extending horizontal and substantially parallel to the top edge, the left slot disposed on the left side portion between a second and third concave line segment members of the four concave line segment members of the left side portion; and a right slot extending horizontal and substantially parallel to the top edge, the right slot disposed on the right side portion between a second and third concave line segment members of the four concave line segment members of the right side portion.

Type IIIB—The Type III nasal dilator is further adapted whereby the left and right at least four concave line segment members consists of four concave line segment members respectively on the left side portion and the right side portion; a left extended-slot extending horizontal and substantially parallel to the top edge, the left extended-slot disposed on the left side portion between a second and third concave line segment members of the four concave line segment members of the left side portion; and a right extended-slot extending horizontal and substantially parallel to the top edge, the right extended-slot disposed on the right side portion between a second and third concave line segment members of the four concave line segment members of the right side portion.

Type IIIC—The Type III nasal dilator is again adapted whereby the left and right at least four concave line segment members consists of five concave line segment members respectively on the left side portion and the right side portion; the left side portion further comprising a first slot disposed between a second and a third concave line segment member and a second slot disposed between the third concave line segment member and a fourth concave line segment member; and the right side portion further comprising a first slot disposed between a second and a third concave line segment member and a second slot disposed between the third concave line segment member and a fourth concave line segment member.

These aforementioned embodiments, and other embodiments are further described in the detailed description below and those skilled in the art will appreciate the benefits and improvements over the state of the current art.

### DRAWING

FIG. 1 is an offset frontal view of a first embodiment of the present invention.

FIG. 2 is an offset frontal view of a second embodiment of the present invention

FIG. 3 is an offset frontal view of a third embodiment of the present invention.

FIG. 4 is a front view of the embodiment of FIG. 1.

FIG. 5 is a front view of the embodiment of FIG. 2.

FIG. 6 is a font view of the embodiment of FIG. 3.

FIG. 7 is an offset frontal view of a shoe having a strip device according to one embodiment of the present invention placed thereon.

FIG. 8 is a rear elevation of a shoe having a strip device according to one embodiment of the present invention placed thereon.

FIG. 9 is a bottom view of a fourth embodiment of a strip device according to the present invention.

FIG. 10 is a rear view of the fourth embodiment of a strip device.

FIG. 11 is a top view of the fourth embodiment of a strip device.

FIG. 12 is a front view of the fourth embodiment of a strip device.

FIG. 13 is a right side view of the fourth embodiment of a strip device.

FIG. 14 is an offset front view of the fourth embodiment of a strip device.

FIG. 15 is a bottom view of a fifth embodiment of a strip device.

FIG. 16 is a rear view of the fifth embodiment of a strip device.

FIG. 17 is a top view of a fifth embodiment of a strip device.

FIG. 18 is a front view of a fifth embodiment of a strip device.

FIG. 19 is a right side view of a fifth embodiment of a strip device.

FIG. 20 is an offset front view of a fifth embodiment of a strip device.

FIG. 21 is a bottom view of a sixth embodiment of a strip device.

FIG. 22 is a rear view of a sixth embodiment of a strip device.

FIG. 23 is a top view of a sixth embodiment of a strip device.

FIG. 24 is a front view of a sixth embodiment of a strip device.

FIG. 25 is a right side view of a sixth embodiment of a strip device.

FIG. 26 is an offset front view of a sixth embodiment of a strip device.

FIG. 27 is a bottom view of a square patch embodiment of a strip device.

FIG. 28 is a top view of the square patch embodiment of a strip device.

FIG. 29 is a front view of the square patch embodiment of a strip device.

FIG. 30 is an offset front view of the square patch embodiment of a strip device.

FIG. 31 is a right side view of the square patch embodiment of a strip device.

FIG. 32 shows the square patch in one environment of use.

FIG. 33 is a back view of an alternate design for the sixth embodiment of a strip device.

FIG. 34 is a front view of the alternate design of FIG. 33.

FIG. 35 is a top view of the alternate design of FIG. 33.

FIG. 36 is an offset front view of the alternate design of FIG. 33.

FIG. 37 shows the sixth embodiment in one possible environment of use.

### DESCRIPTION OF THE INVENTION

Possible preferred embodiments will now be described with reference to the drawings and those skilled in the art will understand that alternative configurations and combinations of components may be substituted without subtracting from the invention. Also, in some figures certain components are omitted to more clearly illustrate the invention.

The present invention hereby incorporates by reference as if fully set out herein co-pending U.S. utility patent application Ser. No. 14/169,466 filed on 2014 Jan. 31 by the common inventor.

In the various figures of the drawing some elements are omitted for clarity. In particular, in a given figure, a plurality of similar layers having a similar shape and configuration, a unique reference number does not necessarily reference each individual feature of each layer. Those skilled in the art will appreciate that the similar features share the same reference number and need not be repeatedly called out and otherwise clutter the figure.

In various preferred embodiments of the present invention a strip device, configured to enhance visual safety, or configured to deliver medicaments directly to the skin, or both, consist of a compound member having a plurality of layers. In one embodiment, as described in co-pending U.S. utility patent application Ser. No. 14/169,466 filed on 2014 Jan. 3, the strip device configures to a nasal dilator consisting of a two or three layer structure termed a constructed truss. One layer includes an acrylic based adhesive layer having an adhesive on one side and a resilient member attached to the opposite side. The nasal dilator has moisture resistant properties and can be made from reflective material.

The present invention adapts this nasal dilator and re-configures the strip device to serve as a nasal dilator with additional capabilities in one contemplated embodiment. In other contemplated embodiments, the strip device is adapted for use as a reflective device to improve safety and can, therefore, be applied to other areas of a wearer (either directly on the skin, or alternatively, on clothing, apparel, gear, footwear, and the like of the wearer—and has a much larger application than as a nasal dilator.

In the various alternative contemplated and preferred embodiments of the present application, one suitable reflective material is a customized coated material that contains a reflective ink. Alternatively, the layer can be a self-illuminating layer having at least one, but preferably, a plurality of LED lights with an ambient light charging system that powers the strip. Reflective, or alternatively self-illumination, features will act as both a safety and breathing function for the device. The reflection or self-illumination will occur from approaching light from vehicles, ambient light, or any other object that shines light in the direction of the nose. It will light up solid colors or imprints including logos. The reflective ink will be applied to the top layer of the material, underneath the flexor.

Another feature of the product is moisture resistance. Any layer, but particularly the adhesive layer is a customized coated material (such as polyethylene) that contains a moisture resistant barrier to prevent liquids from getting underneath the skin-contacting side of the dilator. The barrier will be impervious to sweat, rain, mud or snow, keeping the device in tact and functioning if moisture is applied.

One particular advantage of the preferred embodiments of the present invention using two-layers consisting of an adhesive layer and a resilient layer is the ability of the wearer to better position the strip in a desired position on clothing, gear, footwear, caps, or directly on the skin. When used in direct contact with a wearer's skin, the present invention enables the wearer to experience more comfort over usage time. Further, the position of the flexor and the flared ends of the material provide better bond to the skin to ensure better hold and performance.

The resilient member in many preferred embodiments is a solid, one-piece member that is designed to evenly distribute and otherwise better adhere the strip to the targeted area of adhesion. The design also fits a wide variety of flat or curved shapes (such as the toe or heel of a shoe, a curved face of a helmet, a flat area of a garment, on a dog collar, etc.) and helps keep the strip from lifting up.

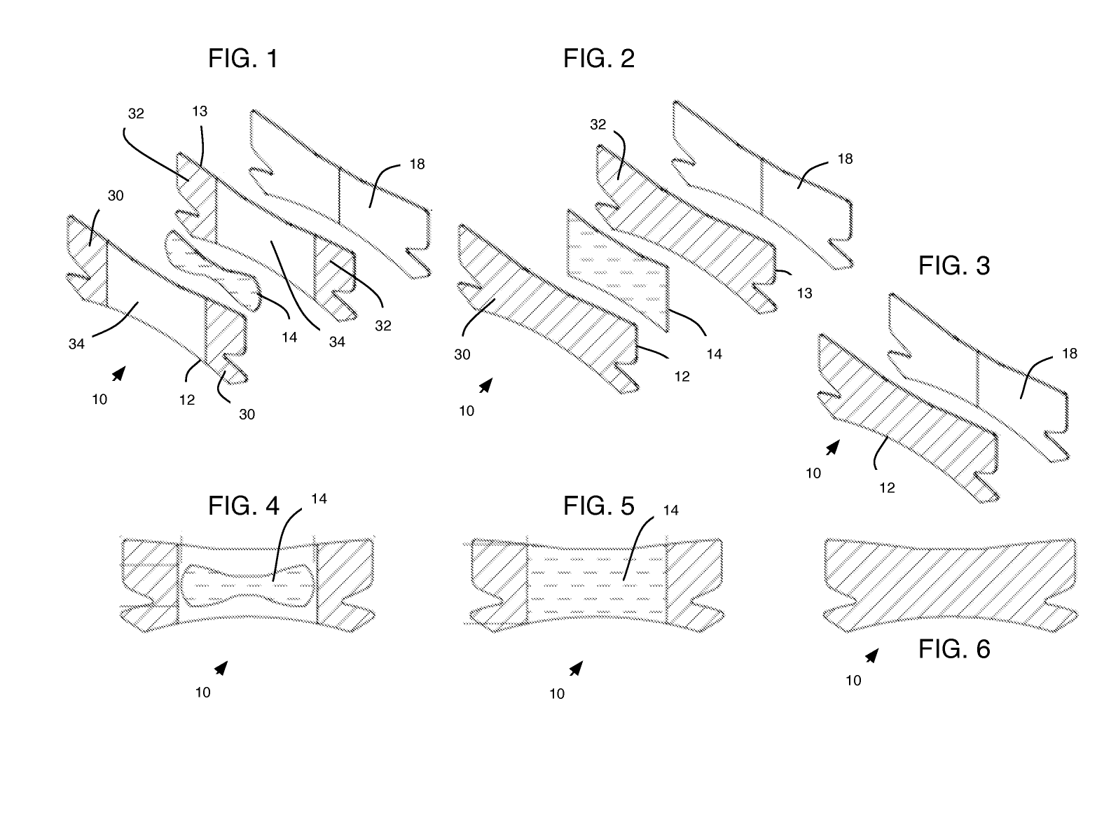

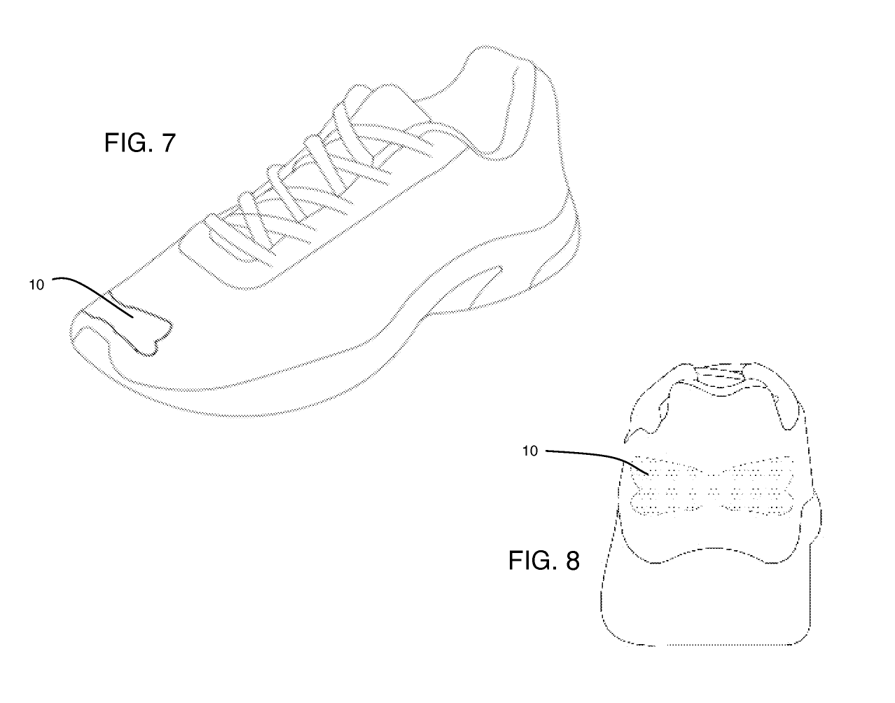

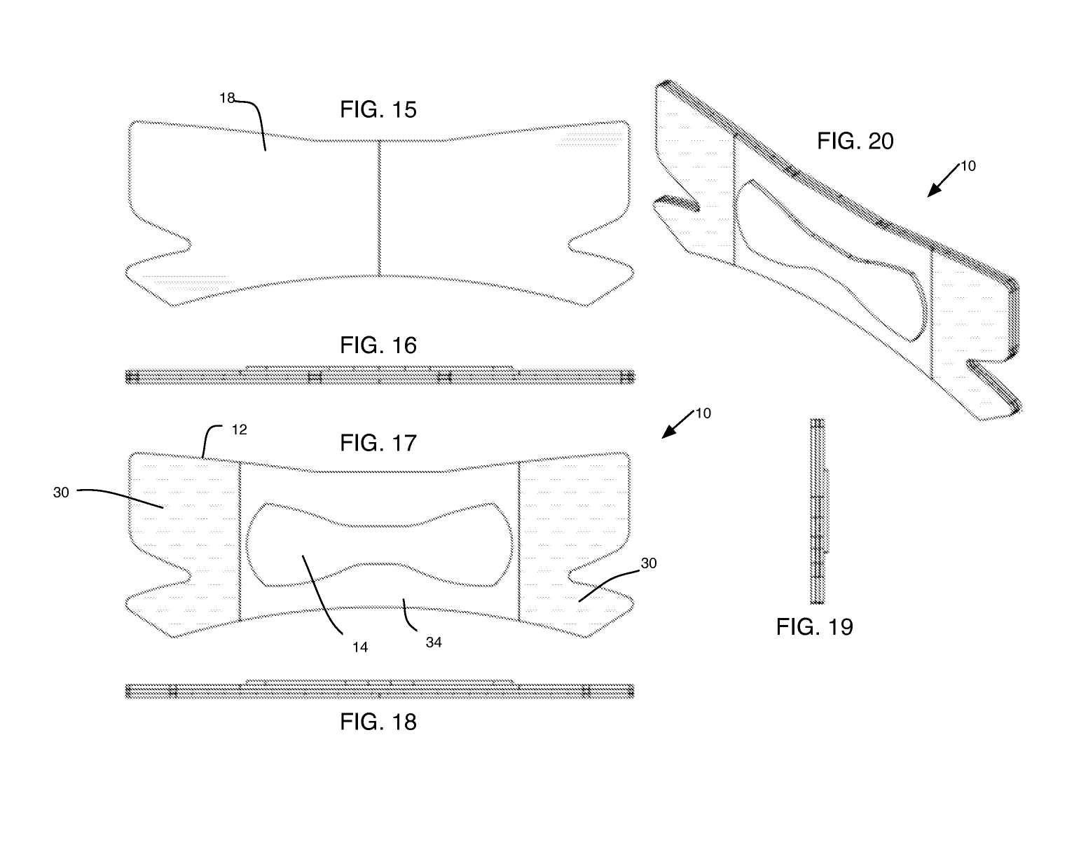

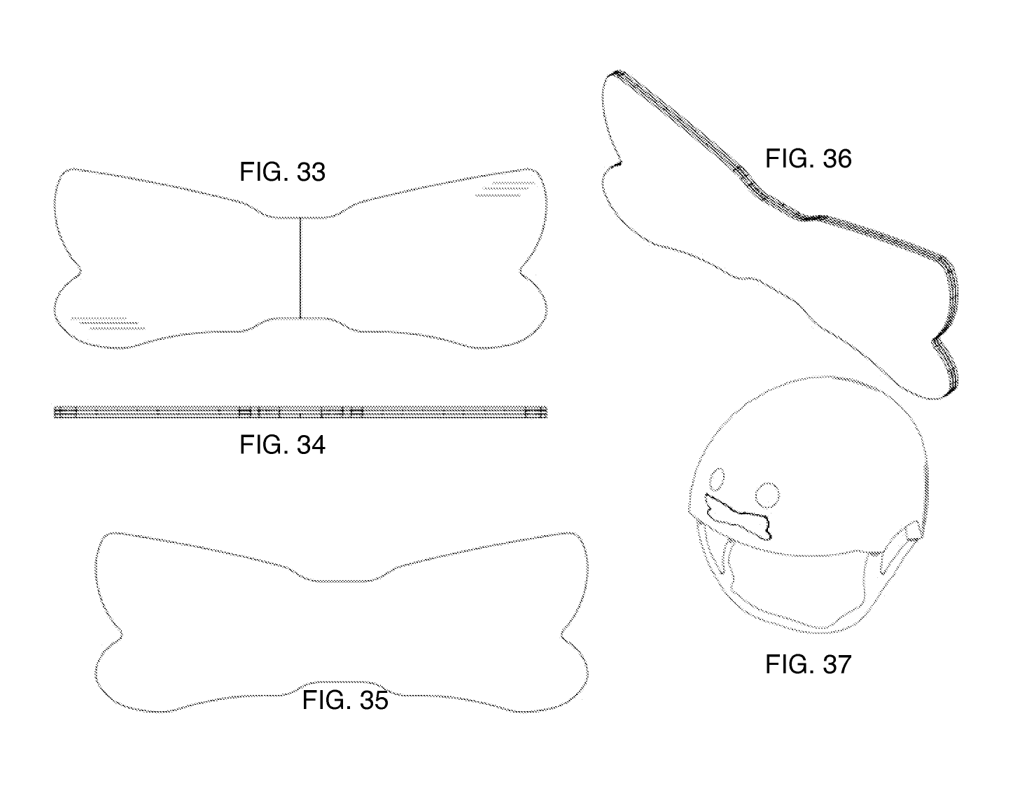

The various embodiments of the present invention, as illustrated FIGS. 1, 2, 4, 5, and 7-37, describe a strip device 10, which is a constructed truss assembly comprising or consisting of at least one layer, and more specifically a plurality of layers comprising a first layer 12 comprising a first-layer elastic member 30 covering at least a portion of the first layer, a second layer 13 comprising a second-layer elastic member 32 covering at least a portion of the second layer, a flexor layer 14 disposed on the first layer, an adhesive coating a back side of the second layer, and an adhesive backing layer 18 removably coupled to the adhesive.

A constructed truss, such as the strip device 10 of the present invention, is an assembly of one or more distinct layers, each layer contributing some desired properties to the strip device. A constructed truss has a normally substantially planar state, the constructed truss comprises a plurality of layers: The layers comprising an adhesive backing layer; an adhesive; a resilient layer coupled to the adhesive layer; and a cover layer coupled to the adhesive layer whereby the resilient layer is disposed between the cover layer and the adhesive layer.

With specific reference to FIGS. 1 and 4, a first embodiment of the present invention includes a strip device 10 consisting of four layers: a first layer 12, a flexor 14, a second layer 13, and an adhesive backing 18. An adhesive is applied to the backside of the second layer. The first layer consists of a first-layer elastic member 30 consisting of two portions separated by an intermediate clear portion 34. The second layer 13 mimics the first layer and, accordingly, comprises a second-layer elastic member 32 consisting of two portions separated by an intermediate clear portion 34. A flexor 14, which arranges in a flat plane, but configured to flex, bend, and otherwise deform, resiliently re-forming into the flat plane. In this embodiment, the clear portion of the first layer enables the flexor to be seen therethrough. The flexor further comprises an optional reflective ink deposited on the top side of the flexor or a self-illuminating feature.

Similarly, the embodiment of FIGS. 2 and 5 show a strip device 10 consisting of four layers: a first layer 12, a flexor 14, a second layer 13, and an adhesive backing 18. An adhesive is applied to the backside of the second layer. In this embodiment, however, the first layer 12 includes an elastic member 30 that makes up the entire first layer. The flexor 14 is disposed between the first and second layers. The flexor 14 is rectangular, as opposed to the more bow-tie shaped flexor described in the first embodiment, above. The second layer includes a second-layer elastic member 24 making up the entire second layer.

FIGS. 3 and 6 illustrate a third embodiment of a strip device comprising only two layers, a first reflective or self-illuminating layer includes a reflective ink or self-illuminating LED device deposited on a first side and an adhesive deposited on an opposite, second side. The second layer comprises a peel-away backing layer.

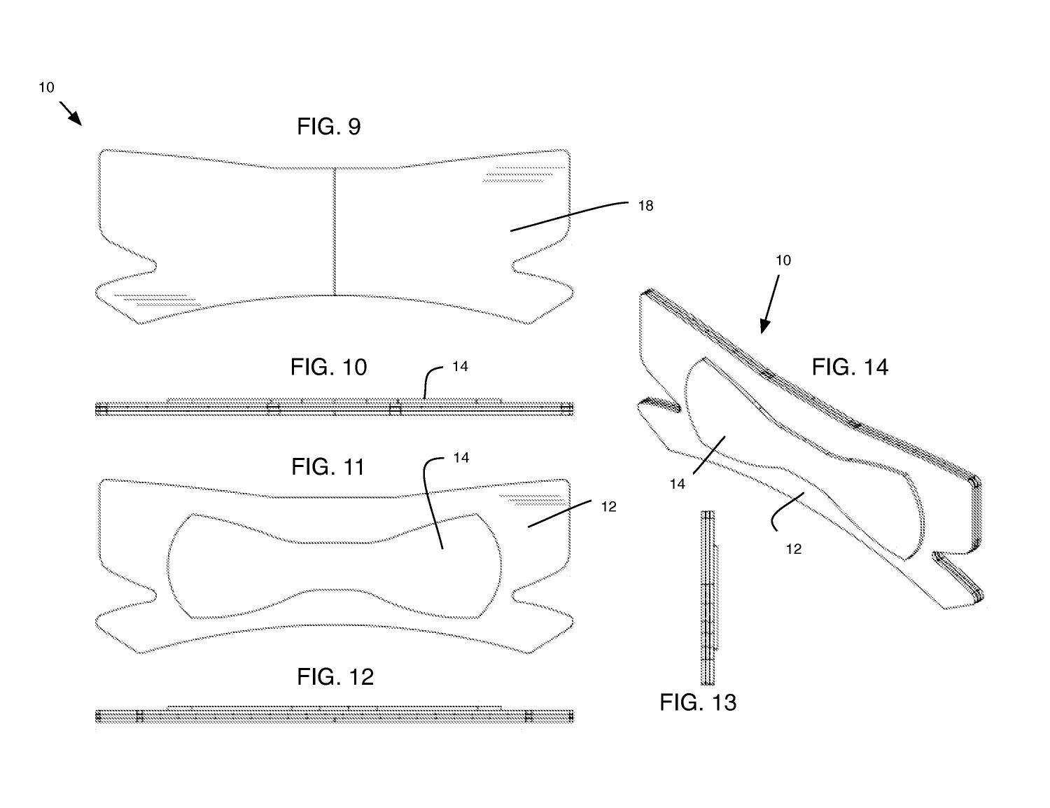

With particular reference to FIGS. 9-14, which show a fourth embodiment wherein the plurality of layers are arranged slightly differently from the first and second embodiments. Specifically, the flexor 14 is placed on top of the first layer 12, the first layer is placed on top of the second layer 13 and the adhesive backing layer is under the second layer 18. Again, the flexor 14 optionally includes a reflective (or self-illuminating) top portion consisting of reflective ink (or self-illuminating LED light device) deposited thereon. Here, the flexor is a bow-tie shape.

FIGS. 15-20 show a fifth embodiment, which is similar to the fourth embodiment whereby the flexor 14 is placed on top of the first layer 12. But the first layer further includes an elastic member 30 having two portion separated by a clear center. Like the fourth embodiment, the second layer 13 consists of an elastic member. Again, the flexor 14 optionally includes a reflective top portion consisting of reflective ink (or, alternatively, a self-illuminating device) deposited thereon. Here, the flexor is a bow-tie shape. The fourth layer is the peel-away adhesive backing.

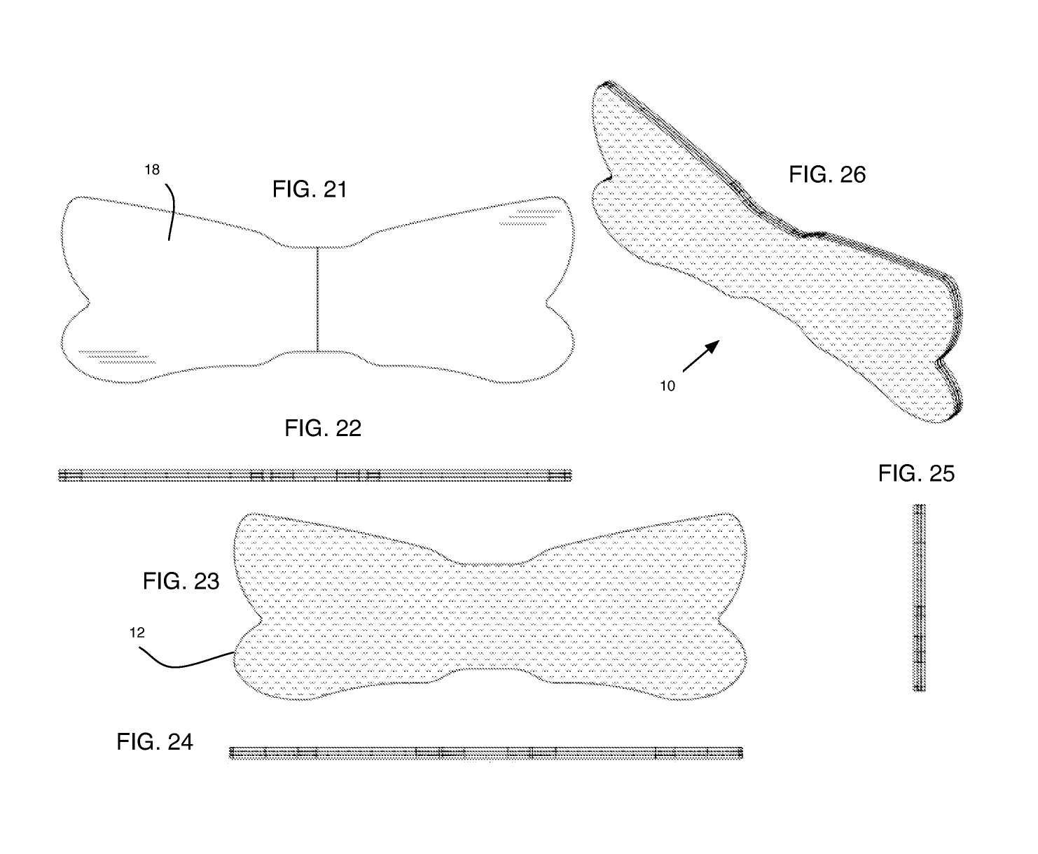

Omitting the flexor, the embodiment of FIGS. 21-26, 33-36, a sixth embodiment having an overall shape of the strip device described as a butterfly shape. Here, a first layer includes a reflective ink or, alternatively, a self-illuminating LED-device. A second layer consists of an elastic member and a truss layer includes a second elastic member. The fourth layer is the adhesive backing.

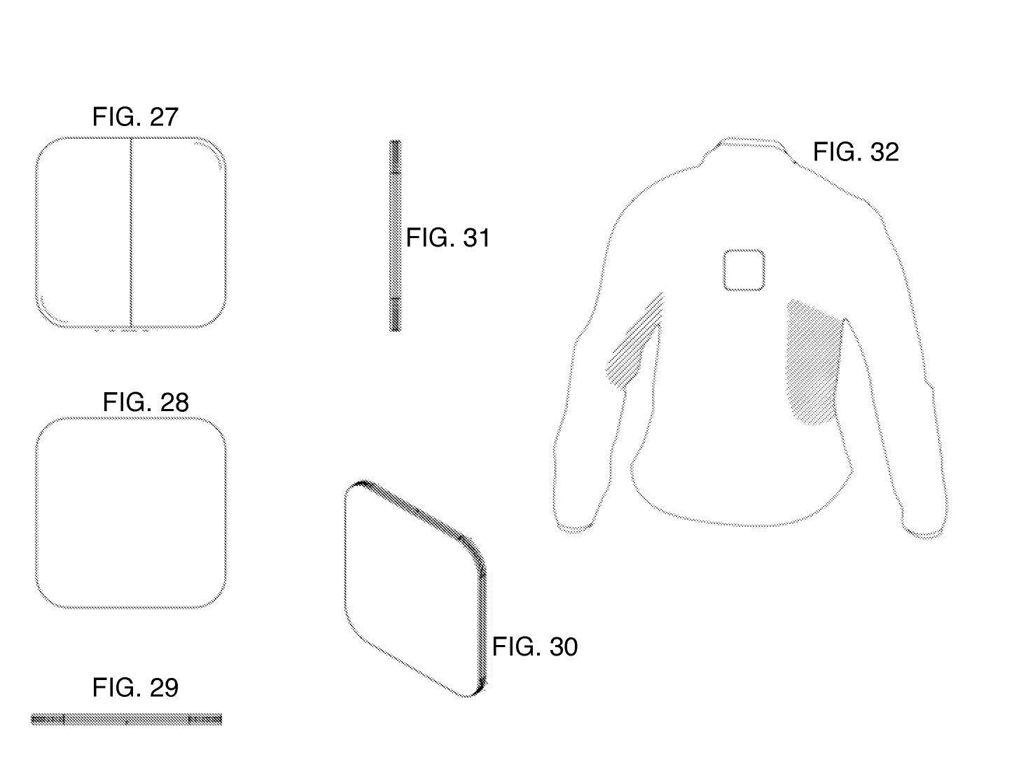

An alternate shape of the sixth embodiment is a square patch design as illustrated by FIGS. 27-32. Here, a first layer includes a reflective ink or, alternatively, a self-illuminating LED-device. A second layer consists of an elastic member and a truss layer includes a second elastic member. The fourth layer is the adhesive backing.

Obviously, there are a myriad of uses of the various preferred embodiments of the present invention. The reflective or, alternatively, a self-illuminating properties of the strip device make it well adapted for decorative or safety uses. Some contemplated uses include functional safety device worn on clothing, shoes, or helmets, for example.

The truss member, in certain embodiments includes a reflective top layer or a self-illuminating top layer (alternatively) consisting of a reflective ink or a self-illuminating LED. In these certain embodiments, the first layer is a clear layer. In other embodiments, the first layer is a reflective or, alternatively, a self-illuminating, layer comprising a top layer of a reflective ink deposited on a PET, or similar, material.

In all of the foregoing preferred embodiments of the present invention. The strip device consists of a layered truss. One suitable arrangement of layers includes a first layer consisting of the cover, a second layer consisting of a resilient member, a third layer consisting of an adhesive layer, and a fourth layer consisting of an adhesive backing layer. Other contemplated embodiments include a first layer consisting of a resilient member and a second layer consisting of an adhesive layer. The first, cover layer further comprises a compound layer comprising any combination, alone or together, including a water-resistant layer, a reflective layer, a color layer, or a medicament-infused layer.

Some contemplated medicaments include common over-the-counter medicines to combat fatigue, colds, flu, and common allergies, for example. Other medicaments include menthol/camphor and other active ingredients found commonly in over-the-counter nasal sprays, for example.

The various embodiments of the present invention contemplate alternative modes of illuminating the strip device 10 to provide higher-visibility in low-light conditions. One alternative is a self-illuminating mode or version which requires either an on-board power source or a power storage device that converts light into electricity, thereby a natural or electrical light source would charge the strip resulting in the illumination of the device to last for several days, for example. A second alternative is reflective whereby an outside light source is simply reflected off a highly reflective layer (or coating). Persons having ordinary skill in the art will readily understand currently commercially available products that provide reflective or self-illumination as contemplated in the various embodiments of the present invention.

Contemplated adhesive materials are either applied to a singular layer known as spun lace, or on one side, depending on the structure of the part. The adhesive is either medical grade if it applies to the skin or a more aggressive industrial grade adhesive for use to bond the strip to articles including fabric, metal, plastic or rubber, for example. Again, persons having ordinary skill in the art will readily understand currently commercially available products that provide adequate adhesive properties.

One contemplated flexor material is an about 1 to 2-millimeter thick polyurethane or polyethylene, for example.

In the various contemplated and preferred embodiments, the first layer or cover layer can consist of PET (clear or colored, but preferably clear), or a non-woven latex-free material (colored to any desired color or pattern, but preferably to a solid, no-pattern tan). The resilient layer consists of a 10-mil PET flexor. One suitable adhesive is a latex-free adhesive. And, the backing layer is a common paper backing material as would be well understood in the art. Other materials and combinations of materials, including color, texture, and pattern, are also contemplated.

In various preferred embodiments some contemplated medicaments include active components having a concentration of about 20 percent. One suitable medicament includes a hydrogel. One particular embodiment of this includes a nose strip or patch device. The hydrogel is a water-based material consisting of a network of polymers, and provides a cooling affect and other medicinal benefits. In this application the strip/patch will contain menthol, herbal, or OTC (over-the-counter) active ingredients that will absorb into the skin, particularly adapted for use as an external strip placed on the nose of the wearer. In this way, the medicament will absorb through the nose and will help reduce swelling of the nasal membranes, common from colds and stuffy nasal passages, and help reduce mucus build up in the nasal cavities. A resilient truss member may be added to the embodiment for added benefit, to help open the nose more completely.

Another contemplated medicament includes the active ingredient found in nasal sprays, Oxymetazoline, which induces vasoconstriction. Coated on or into one of the layers, such as into PE or Urethane material-layer. This can be combined with the structured truss or any of the aforementioned embodiments, thus enabling the user or wearer of a strip so constructed to achieve maximum relief from congestion in the deeper regions of the nasal passageways, for example.

Yet another contemplated embodiment includes providing a strip device having a layer infused or coated with an over-the-counter sleep active ingredient. This medicament can be included in a strip device configured to be placed or worn in direct contact with the skin, particularly adapted for use over (externally) the nose. The active medicament will absorb into the nasal cavities/blood stream, providing relief for sleep and reducing or eliminating the need for oral applications. The spring element in the nasal strip opens the nasal passages, and together the product will reduce or eliminate snoring to help improve breathing and sleep benefits.

The medicaments can be time-release formulations.

Although the invention has been particularly shown and described with reference to certain embodiments, it will be understood by those skilled in the art that various changes in form and detail may be made without departing from the spirit and scope of the invention.

I claim:

1. A strip device comprising:
a plurality of layers comprising
a first layer comprising a first-layer elastic member covering at least a portion of the first layer;
a second layer comprising a second-layer elastic member covering at least a portion of the second layer;
a flexor layer disposed on the first layer;
an adhesive disposed on a back side of any at least one of the plurality of layers; and
an adhesive backing removably coupled to the adhesive.

2. The strip device of claim 1 further comprising:
the first layer comprises a reflective ink deposited on at least a portion of the first layer.

3. The strip device of claim 1 further comprising:
the first layer comprises a self-illuminating device comprising at least one LED light and a power-storage device deposited on at least a portion of the first layer.

4. The strip device of claim 2 wherein:
the reflective ink is deposited on a first portion and a second portion with a clear portion disposed intermediate to the first and second portions.

5. The strip device of claim 1 further comprising:
the first layer comprises a clear portion.

6. The strip device of claim 5 wherein:
the clear portion comprises the entire first layer.

7. The strip device of claim 1 further comprising:
the first layer comprises a first-layer elastic member disposed on at least a portion of the first layer.

8. The strip device of claim 7 wherein:
the first-layer elastic member comprises a first portion and a second portion with a clear portion disposed intermediate to the first and second portion.

9. The strip device of claim 1 further comprising:
the second layer comprises a second layer elastic member disposed on at least a portion of the second layer.

10. The strip device of claim 7 wherein:
the second-layer elastic member comprises a first portion and a second portion with a clear portion disposed intermediate to the first and second portion.

11. The strip device of claim 1 configured to be a nasal dilator wherein:
the truss member comprises at least one resilient member being symmetrical with respect to its centerline, the at least one resilient member forming a substantially ovoid profile, the ovoid profile being a first height at the centerline and converging to common left side intersection point and a common right side intersection point with respect to the centerline.

12. The strip device of claim 1 wherein the first layer comprises:
a coating consisting of polyethylene applied to a surface of the first layer.

13. The strip device of claim 1 wherein:
any at least one layer of the plurality of layers further comprises a medicament.

14. The strip device of claim 13 wherein:
the medicament comprises a 20-percent solution including Oxymetazoline.
